(19) United States
(12) Patent Application Publication
(10) Pub. No.: US 2012/0134879 A1
(43) Pub. Date: May 31, 2012

(54) ULTRAVIOLET DISINFECTION PROCESS AND APPARATUS

(76) Inventor: Mohamed Tarifi, Derby, CT (US)

(21) Appl. No.: 12/955,679

(22) Filed: Nov. 29, 2010

(51) Int. Cl. A61L 2/10; B01J 19/12

(52) U.S. Cl. 422/24; 422/186.3

(57) ABSTRACT

A water/wastewater treatment apparatus combines photo catalytic oxidation (PCO) with ultraviolet C (UVC) disinfection, resulting in significantly increasing organism kills from up to log 2 to up to log4. In addition, PCO destruction of organic compounds and its super hydrophilicity reduces or eliminates the potential of quartz sleeve fouling. The quartz sleeve of the present invention includes a nano-layer of titanium dioxide (TiO₂) on the outer surface thereof. An optimum amount of UVC photons transmits through the quartz sleeve and excites the TiO₂ layer, triggering a powerful PCO process that kills organisms on contact and breaks down volatile organic compounds (VOCs) to carbon dioxide (CO₂) and water (H₂O). The majority of UVC photons will penetrate the quartz sleeve and TiO₂ layer to disperse throughout the water/wastewater flow, killing organisms on contact.

### BACKGROUND OF THE INVENTION

The present invention relates to water/wastewater purification systems and, more particularly, to an ultraviolet “C” (UVC) disinfection process and apparatus that increases organism kill by up to log2 and reduces or eliminates quartz sleeve fouling.

UVC disinfection is a natural, non-chemical method of treatment of water/wastewater. UVC disinfection works, for example, by destroying bacteria, viruses and molds by altering their DNA in a process called thymine dimerization. UVC disinfection may find application in wastewater treatment, drinking water treatment, private potable water purification, groundwater remediation and the like.

Currently, UVC disinfection is primarily carried out by UVC lamps enclosed in quartz sleeves. With the current process, UVC disinfection achieves up to log2 organism kill and disinfection is degraded by fouling of the quartz sleeve, requiring constant cleaning of the sleeve by mechanical and/or chemical agents.

As can be seen, there is a need for an apparatus and method for UVC disinfection that increases organism kill by up to log2 while minimizing or eliminating fouling of the quartz sleeve housing the UVC lamp.

### SUMMARY OF THE INVENTION

In one aspect of the present invention, a water/wastewater treatment apparatus comprises an ultraviolet C (UVC) lamp; a quartz sleeve encasing the UVC lamp; and a nano coating of Titanium Dioxide (TiO₂) on the exterior of the quartz sleeve.

In another aspect of the present invention, a water/wastewater treatment apparatus comprises a ultraviolet C (UVC) lamp; a quartz sleeve encasing the UVC lamp; and a nano coating of TiO₂ on the exterior of the quartz sleeve, wherein the nano coating of TiO₂ absorbs between about 2 percent to about 10 percent of UVC photons generated by the UVC lamp.

In a further aspect of the present invention, a method for eliminating fouling of a quartz sleeve housing a ultraviolet C (UVC) lamp during UVC disinfection of water/wastewater passing over the quartz sleeve, the method comprises coating the quartz sleeve with a nano coating of TiO₂, wherein the nano coating has a thickness adapted to allow between about 2 percent to about 10 percent of UVC photons generated by the UVC lamp is absorbed to trigger a photo catalytic oxidation (PCO) process.

These and other features, aspects and advantages of the present invention will become better understood with reference to the following drawings, description and claims.

### BRIEF DESCRIPTION OF THE DRAWINGS

FIG. 1 is a side view of a UVC/PCO disinfection apparatus according to an embodiment of the present invention;

FIG. 2 is a cross-sectional view of the UVC/PCO disinfection apparatus, taken along line 2-2 of FIG. 1; and

FIG. 3 is a cross-sectional view of the UVC/PCO disinfection apparatus, taken along line 3-3 of FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides a water/wastewater treatment apparatus that combines photo catalytic oxidation (PCO) with ultraviolet C (UVC) disinfection, resulting in significantly increasing organism kill from up to log 2 to up to log4. In addition, PCO destruction of organic compounds and its super hydrophilicity reduces or eliminates the potential of quartz sleeve fouling. The quartz sleeve of the present invention includes a nano-layer of TiO₂ on the outer surface thereof. An optimum amount of UVC photons transmits through the quartz sleeve and excites the TiO₂ layer, triggering a powerful PCO process that kills organisms on contact and breaks down volatile organic compounds (VOCs) to carbon dioxide (CO₂) and water (H₂O). The majority of UVC photons will penetrate the quartz sleeve and TiO₂ layer to disperse throughout the water/wastewater flow, killing organisms on contact.

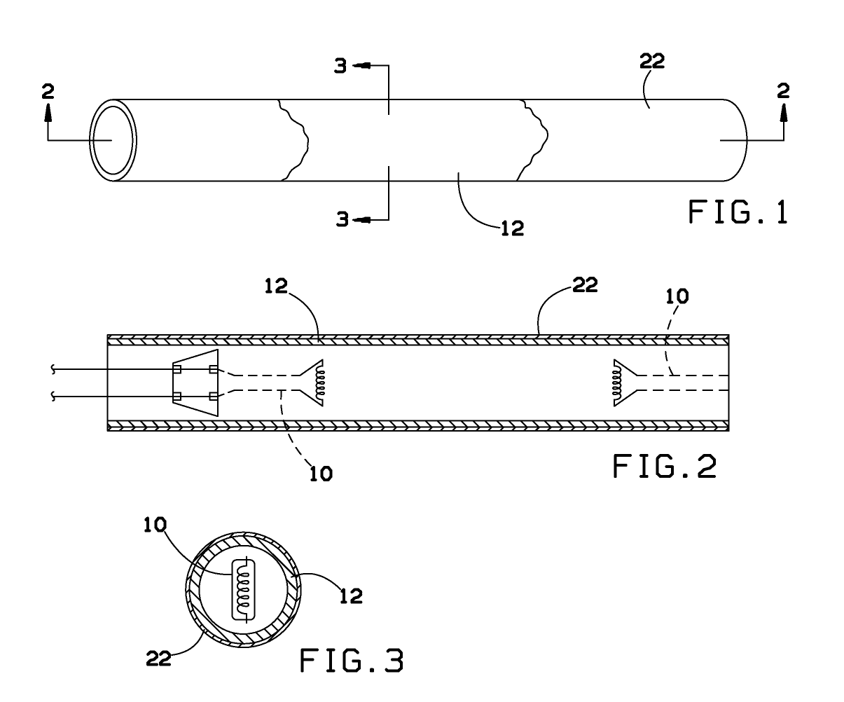

Referring to FIGS. 1 through 3, a UVC lamp 10 may be encased in a quartz sleeve 12. The UVC lamp 10 may be powered via a power line 14. In use, the quartz sleeve 12, having the UVC lamp 10 therewithin, may be placed in a water flow pipe, open channel, or the like. The quartz sleeve 12 is coated by a nano layer 22 of TiO₂ on an outer surface thereof.

Embodiments of the present invention may affect an optimum distribution of UVC photons between the PCO (created by the nano coating 22 of TiO₂ on the quartz sleeve 12) and the UVC process to maximize the effectiveness of both processes. Selecting the proper thickness of the nano coating 22 of TiO₂ on the quartz sleeve 12 may optimize the amount of UVC photons absorbed on the surface of the quartz sleeve 12 while allowing a significant balance of UV photons to carry out an effective UVC disinfection.

The super hydrophilicity of the TiO₂ helps maintain a thin layer of water/wastewater around the quartz sleeve. Fouling of the quartz sleeve may be caused by inorganic contaminants complexing with organic compounds before depositing on the surface and causing fouling. The breakdown of organic compounds by the PCO process coupled with the cleansing velocity of the thin layer of water/wastewater encasing the sleeve may interrupt the complexation of inorganic compounds with organic compounds and prevent any possible deposit of fouling material on the sleeve.

The nano coating 22 of TiO₂ on the quartz sleeve 12 may be applied with an electrostatic gun or similar device. By coating the quartz sleeve 12 with TiO₂, the outside area of the sleeve may be turned into a semiconductor. When immobilized TiO₂ is subjected to UVC photons, it triggers a powerful PCO process that destroys organism's DNA and breaks down organic compounds to CO₂ and H₂O. In addition, UVC transmitted through the sleeves may continue to kill organisms on contact.

The nano coating 22 of TiO₂ on the quartz sleeve 12 may absorb a small percent of UVC photons. For example, the TiO₂ nano coating 22 may absorb from about 2 to about 10 percent of the UVC photons, allowing the majority of the UVC photons to penetrate the sleeve and disperse throughout the water/wastewater and destroy organisms on contact. The combined effect of PCO and UVC increases the disinfection level from up to log2 to up to log4 and the mitigation of sleeve fouling precludes any degradation of disinfection performance.

Embodiments of the present invention can be applied to the field of air and water purification. The TiO₂ nano coating 22 can be immobilized on any surface, thereby facilitating unique applications in air and water purifications. For example, a) TiO₂ may be immobilized on an activated carbon filter which would trap toxic organics and allow PCO to break it down and eliminate the production of intermediates, such as formaldehyde and acetyl aldehyde; b) PCO and UVC can replace current activated carbon filters used in homes. These filters can accomplish the opposite of what they are designed for, when organic compounds get absorbed in the carbon media and can become a growth media for bacteria; c) by combining UVC and PCO, indoor air in homes, buildings, airplanes, submarines, auto cabins, and the like can be purified; and d) by combining UVC and PCO, the design and effectiveness of point of use water treatment can be significantly improved.

It should be understood, of course, that the foregoing relates to exemplary embodiments of the invention and that modifications may be made without departing from the spirit and scope of the invention as set forth in the following claims.

What is claimed is:

1. A water/wastewater treatment apparatus comprising:
a ultraviolet C (UVC) lamp;
a quartz sleeve encasing the UVC lamp; and
a nano coating of titanium dioxide (TiO₂) on the exterior of the quartz sleeve.

2. The water/wastewater treatment apparatus of claim 1, wherein the nano coating of TiO₂ absorbs between about 2 percent to about 10 percent of UVC photons generated by the UVC lamp.

3. The water/wastewater treatment apparatus of claim 1, wherein the nano coating of TiO₂ causes photo catalytic oxidation of organic compounds.

4. The water/wastewater treatment apparatus of claim 3, wherein such treatment apparatus has up to a log4 organism kill.

5. The water/wastewater treatment apparatus of claim 1, wherein the nano coating of TiO₂ eliminates fouling of the quartz sleeve during use thereof.

6. A water/wastewater treatment apparatus comprising:
a ultraviolet C (UVC) lamp;
a quartz sleeve encasing the UVC lamp; and
a nano coating of titanium dioxide (TiO₂) on the exterior of the quartz sleeve,
wherein the nano coating of TiO₂ absorbs between about 2 percent to about 10 percent of UVC photons generated by the UVC lamp.

7. The water/wastewater treatment apparatus of claim 6, wherein:
the photo catalytic oxidation results in the destruction of organic compounds;
UVC photons passing through the quartz sleeve causes UVC disinfection of the water/wastewater; and
the water/wastewater treatment apparatus has up to a log4 organism

8. The water/wastewater treatment apparatus of claim 6 eliminates fouling of the quartz sleeve during use thereof.

9. A method for eliminating fouling of a quartz sleeve housing an ultraviolet C (UVC) lamp during UVC disinfection of water/wastewater passing over the quartz sleeve, the method comprising:
coating the quartz sleeve with a nano coating of titanium dioxide (TiO₂), wherein the nano coating has a thickness adapted to allow between about 2 percent to about 10 percent of UVC photons generated by the UVC lamp is absorbed to trigger a photo catalytic oxidation (PCO) process.

10. The method of claim 9, further comprising causing a thin layer of water/wastewater to cling to the outer surface of the quartz sleeve due to the super hydrophilicity of the nano coating of TiO₂.
